(19) United States
(12) Patent Application Publication
(10) Pub. No.: US 2019/0336478 A1
(43) Pub. Date: Nov. 7, 2019

(54) STABLE ORAL LIQUID COMPOSITIONS OF ENALAPRIL

(71) Applicant: HETERO LABS LIMITED, Hyderabad (IN)

(72) Inventors: Parthasaradhi Reddy BANDI, Hyderabad (IN); Khadgapathi PODILE, Hyderabad (IN); Sunil Deviprasad TIWARI, Hyderabad (IN); Prakash SHETIYA, Hyderabad (IN); Satyanarayana Rao PATCHIGOLLA, Hyderabad (IN); Ravi Chandra Gupta CHIDARA, Hyderabad (IN)

(73) Assignee: HETERO LABS LIMITED, Hyderabad (IN)

(21) Appl. No.: 16/403,444

(22) Filed: May 3, 2019

(30) Foreign Application Priority Data
May 4, 2018 (IN) 201841016887

(51) Int. Cl. A61K 31/40; A61K 9/08; A61K 9/00; A61K 47/12; A61K 47/02; A61P 9/04; A61P 9/12

(52) U.S. Cl. CPC A61K 31/40; A61K 9/08; A61K 9/0056; A61P 9/12; A61K 47/02; A61P 9/04; A61K 47/12

(57) ABSTRACT

The present invention relates to pharmaceutical compositions of enalapril, particularly stable oral liquid compositions comprising enalapril, one or more buffering agents and at least one pharmaceutically acceptable excipient.

### CROSS REFERENCE TO RELATED APPLICATIONS

This application claims priority to Indian Patent Application No. 201841016887, filed on May 4, 2018; the disclosure of all of which is hereby incorporated by reference in its entirety.

### FIELD OF THE INVENTION

The present invention relates to stable oral liquid compositions comprising enalapril or its pharmaceutically acceptable salt and one or more pharmaceutically acceptable excipients.

### BACKGROUND OF THE INVENTION

Enalapril is an angiotensin-converting enzyme (ACE) inhibitor. Chemically it is (S)-1-[N-[1-(ethoxycarbonyl)-3-phenylpropyl]-L-alanyl]-L-proline, having empirical formula C₂₀H₂₈N₂O₅ with molecular weight 376.447 g/mol. Its structural formula is:

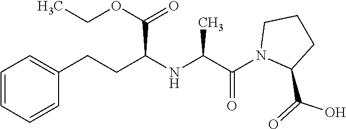 [SMILES: CCOC(=O)[C@H](CCc1ccccc1)N[C@@H](C)C(=O)N1CCC[C@H]1C(=O)O]

Enalapril is used for the treatment of hypertension in adults and children older than one month, symptomatic heart failure and treatment of asymptomatic left ventricular dysfunction.

In US, Enalapril is commercially available as 2.5 mg, 5 mg, 10 mg and 20 mg tablets with brand name Vasotec® from Valeant Pharmaceuticals; as 1 mg/mL powder for oral solution with brand name Epaned Kit® and 1 mg/mL oral solution with brand name Epaned® from Silvergate pharmaceuticals.

U.S. Pat. No. 4,374,829 discloses Enalapril.

U.S. Pat. No. 9,669,008 and U.S. Publication No. 2018/0055821 assigned to Silvergate pharmaceuticals claims oral liquid composition comprising enalapril maleate, citric acid and sodium citrate dihydrate as buffer, wherein the composition is having about 5% w/w of total impurities.

Inventors of the present invention are developing stable oral liquid compositions of enalapril using novel excipients with improved stability.

### SUMMARY OF THE INVENTION

One embodiment of the present invention relates to stable oral liquid compositions of enalapril or a pharmaceutically acceptable salt thereof, one or more buffering agents and at least one pharmaceutically acceptable excipient.

Another embodiment of the present invention relates to stable liquid compositions for oral administration comprising a therapeutically effective amount of enalapril or a pharmaceutically acceptable salt thereof and a buffering agent selected from disodium hydrogen phosphate, glycine, hydrochloric acid, citric acid, glacial acetic acid and sodium acetate trihydrate.

Another embodiment of the present invention relates to stable liquid composition for oral administration comprising 1 mg/mL of enalapril maleate and a buffer comprising 1 mg/mL to 3 mg/mL of citric acid and 0.01 mg/mL to 0.3 mg/mL of disodium hydrogen phosphate.

Another embodiment of the present invention relates to stable liquid compositions for oral administration comprising a therapeutically effective amount of enalapril or a pharmaceutically acceptable salt thereof and a preservative selected from sodium benzoate, potassium sorbate, sorbic acid, methyl paraben and propyl paraben.

Another embodiment of the present invention relates to stable liquid composition for oral administration comprising 1 mg/mL of enalapril maleate, a buffer comprising 1 mg/mL to 3 mg/mL of citric acid and 0.10 mg/mL to 0.30 mg/mL of trisodium citrate and potassium sorbate as preservative.

Another embodiment of the present invention relates to stable liquid composition for oral administration comprising 1 mg/mL enalapril maleate and a buffer comprising 1.82 mg/mL citric acid and up to 0.15 mg/mL disodium hydrogen phosphate; wherein the composition has less than 1.5% of total impurities after storage for 15 months at 2-8° C.

Another embodiment of the present invention relates to stable liquid composition for oral administration comprising 1 mg/mL enalapril maleate and a buffer comprising 1.82 mg/mL citric acid and 0.15 mg/mL disodium hydrogen phosphate; wherein the composition has less than 5% of total impurities after storage for 6 months at 25° C.

Another embodiment of the present invention relates to method of treating hypertension, heart failure and asymptomatic left ventricular dysfunction by administering said composition to the patient.

### DETAILED DESCRIPTION OF THE INVENTION

The present invention relates to stable oral liquid compositions of enalapril with one or more buffering agents.

The term “enalapril” as used herein according to the present invention includes enalapril in the form of free base, a pharmaceutically acceptable salt thereof, amorphous enalapril, crystalline enalapril or any isomer, derivative, hydrate, solvate, or prodrug or combinations thereof. Preferably, enalapril maleate.

The term “excipient” means a pharmacologically inactive component such as a preservative, a buffering agent, a sweetener, a flavor etc., of a pharmaceutical product. The excipients that are useful in preparing a pharmaceutical composition are generally safe, non-toxic and are acceptable for human pharmaceutical use. Reference to an excipient includes both one and more than one such excipients.

The term “pharmaceutically acceptable” as used herein means that which is useful in preparing a pharmaceutical composition that is generally safe and non-toxic.

The term “composition” or “pharmaceutical composition” as used herein synonymously include liquid dosage forms such as solutions (aqueous and non-aqueous), suspensions, emulsions, syrups, elixirs and the like meant for oral administration, preferably, solutions.

As used in this specification and the appended claims, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

In one aspect, the present invention provides a stable liquid composition for oral administration comprising a therapeutically effective amount of enalapril or a pharmaceutically acceptable salt thereof and one or more buffering agents.

In another aspect, the present invention involves controlling enalaprilat and enalapril diketopiperazine impurities in an acceptable range in the oral liquid composition of enalapril.

Buffering agents according to the present invention include citric acid, disodium hydrogen phosphate, glycine, hydrochloric acid, glacial acetic acid, sodium acetate trihydrate, trisodium citrate, potassium chloride, hydroxymethyl aminomethane, sodium hydroxide, carbonate, bicarbonate and the like and combinations thereof.

In another aspect, the buffering agent is a combination of citric acid anhydrous and disodium hydrogen phosphate.

Excipients of the present invention further comprise sweeteners, flavors and preservatives.

Sweeteners according to the present invention include glucose, fructose, sucrose, xylitol, sucralose, maltitol, lactitol, sorbitol, erythritol, trehalose, maltodextrin, polydextrose, and the like and combinations thereof.

Flavors according to the present invention include orange, peach, pear, peppermint, pineapple, cranberry, grape, grapefruit, guava, hop, lemon, lime, malt, molasses, mixed berry, raspberry, rose, vanilla, wintergreen, spearmint, strawberry, etc and the like and combinations thereof.

Preservatives according to the present invention include sodium benzoate, potassium sorbate, sorbic acid, methyl paraben, propyl paraben and the like and combinations thereof.

In another aspect, the present invention provides a stable liquid composition for oral administration comprising 1 mg/mL of enalapril maleate and a buffer comprising 1 mg/mL to 3 mg/mL of citric acid anhydrous and 0.01 mg/mL to 0.3 mg/mL of disodium hydrogen phosphate.

In another aspect, the present invention provides a stable liquid composition for oral administration comprising 1 mg/mL of enalapril maleate and a buffer comprising 1.82 mg/mL of citric acid anhydrous and 0.15 mg/mL of disodium hydrogen phosphate.

In another aspect, the present invention provides a stable liquid composition for oral administration comprising 1 mg/mL of enalapril maleate and a buffer comprising 1.82 mg/mL of citric acid anhydrous and 0.07 mg/mL of disodium hydrogen phosphate.

In another aspect, the present invention relates to stable liquid compositions for oral administration comprising a therapeutically effective amount of enalapril or a pharmaceutically acceptable salt thereof and a preservative selected from sodium benzoate, potassium sorbate, sorbic acid, methyl paraben and propyl paraben.

In another aspect, the present invention relates to stable liquid composition for oral administration comprising 1 mg/mL enalapril maleate and a buffer comprising citric acid in an amount of 1 mg/mL to 3 mg/mL and disodium hydrogen phosphate in an amount of 0.01 mg/mL to 0.3 mg/mL, wherein the composition has less than 1.5% of total impurities after storage for 15 months at 2-8° C.

In another aspect, the present invention relates to stable liquid composition for oral administration comprising 1 mg/mL enalapril maleate and a buffer comprising of 1.82 mg/mL citric acid anhydrous and 0.15 mg/mL disodium hydrogen phosphate; wherein the composition has less than 1.5% of total impurities after storage for 15 months at 2-8° C.

In another aspect, the present invention relates to stable liquid composition for oral administration comprising 1 mg/mL enalapril maleate and a buffer comprising of 1.82 mg/mL citric acid anhydrous and 0.07 mg/mL disodium hydrogen phosphate; wherein the composition has less than 1.5% of total impurities after storage for 15 months at 2-8° C.

In another aspect, the present invention provides a stable liquid composition for oral administration comprising 1 mg/mL of enalapril maleate, a buffer comprising 1 mg/mL to 3 mg/mL of citric acid and 0.10 mg/mL to 0.30 mg/mL of trisodium citrate and potassium sorbate as preservative.

Preferably, the present invention provides a stable liquid composition for oral administration comprising 1 mg/mL of enalapril maleate, a buffer comprising 1.82 mg/mL of citric acid anhydrous and 0.15 mg/mL of trisodium citrate and potassium sorbate as preservative; wherein the composition has less than 2% of total impurities after storage for 3 months at 2-8° C.

In another aspect, the present invention provides a stable liquid composition for oral administration comprising 1 mg/mL enalapril maleate and a buffer comprising citric acid in an amount of 1 mg/mL to 3 mg/mL and disodium hydrogen phosphate in an amount of 0.01 mg/mL to 0.3 mg/mL; wherein the said composition has less than 5% of total impurities after storage for 6 months at 25° C.

In another aspect, the present invention provides a stable liquid composition for oral administration comprising 1 mg/mL enalapril maleate and a buffer comprising of 1.82 mg/mL citric acid and 0.07 mg/mL disodium hydrogen phosphate; wherein the said composition has less than 5% of total impurities after storage for 6 months at 25° C.

Compositions of the present invention are useful in the treatment of hypertension, heart failure and asymptomatic left ventricular dysfunction.

### EXAMPLES

The following examples further describe and demonstrate particular embodiments within the scope of the present invention. The examples are given solely for illustration and are not to be construed as limitations as many variations are possible without departing from spirit and scope of the invention.

### Example—1

| Ingredient | mg/mL |
|---|---|
| Enalapril maleate | 1.00 |
| Citric acid anhydrous | 1.82 |
| Disodium hydrogen phosphate | 0.15 |
| Sucralose | 0.70 |
| Sodium benzoate | 1.00 |
| Starwberry flavor | 0.5 |
| Purified water | q.s |

### Brief Manufacturing Process:

1. Sodium benzoate to part of purified water was added and continuously stirred till clear solution was formed.
2. Citric acid followed by disodium hydrogen phosphate was added to part of purified water and continuously stirred till clear solution was formed.
3. Step 1 and step 2 were continuously stirred till clear solution was formed.
4. Sucralose and flavor were added to step 3 and continuously stirred till clear solution was formed.
5. Enalapril maleate was added to step 4 and continuously stirred till clear solution was formed and final volume was made with purified water.
6. pH of solution (3.0-3.5) was checked and stored at suitable conditions.

TABLE 1
Results of stability evaluation of Enalapril oral liquid prepared from Example 1:

| | | | | Example 1 | | | | |
|---|---|---|---|---|---|---|---|---|
| | Epaned ® | | | (Enalapril maleate oral solution 1 mg/mL) | | | | |
| Related | 25° C./60% RH | | 2-8° C. | | 25° C./60% RH | | 2-8° C. | |
| Substances | 3 months | 6 months | 3 months | Initial | 3 months | 6 months | 3 months | 15 months |
| Enalaprilat | 2.200% | 4.764% | 0.393% | 0.091% | 2.070% | 3.580% | 0.420% | 0.783% |
| (Impurity- C) | | | | | | | | |
| Enalapril | 0.625% | 0.990% | 0.042% | 0.072% | 0.680% | 1.040% | 0.120% | 0.131% |
| Diketopiperazine | | | | | | | | |
| (Impurity - D) | | | | | | | | |
| Total impurities | 2.820% | 5.763% | 0.435% | 1.625% | 2.750% | 4.780% | 0.540% | 0.914% |

Results of table 1 indicates that, composition of example 1 contains less than 1.5% of total impurities when stored at 2-8° C.

### Example—2

| Ingredient | mg/mL |
|---|---|
| Enalapril maleate | 1.00 |
| Glycine | 3.50 |
| Hydrochloric acid | 0.50 |
| Sucralose | 0.70 |
| Sodium benzoate | 1.00 |
| Mixed berry flavor | 0.36 |
| Purified water | q.s |

### Brief Manufacturing Process:

1. Sodium benzoate to part of purified water was added and continuously stirred till clear solution was formed.
2. Glycine followed by hydrochloric acid was added to part of purified water and continuously stirred till clear solution was formed.
3. Step 1 and step 2 were continuously stirred till clear solution was formed.
4. Sucralose and flavor were added to step 3 and continuously stirred till clear solution was formed.
5. Enalapril maleate was added to step 4 and continuously stirred till clear solution was formed and final volume was made with purified water.
6. pH of solution (3.0-3.5) was checked and stored at suitable conditions.

### Example—3

| Ingredient | mg/mL |
|---|---|
| Enalapril maleate | 1.00 |
| Glacial acetic acid | 5.00 |
| Sodium acetate trihydrate | 0.25 |
| Sucralose | 0.70 |
| Sodium benzoate | 1.00 |
| Mixed berry flavor | 0.36 |
| Purified water | q.s |

### Brief Manufacturing Process:

1. Sodium benzoate to part of purified water was added under continuous stirring till clear solution was formed.
2. Glacial acetic acid followed by sodium acetate trihydrate was added to part of purified water and continuously stirred till clear solution was formed.
3. Step 1 and step 2 were continuously stirred till clear solution was formed.
4. Sucralose and flavor were added to step 3 and continuously stirred till clear solution was formed.
5. Enalapril maleate was added to step 4 and continuously stirred till clear solution was formed and final volume was made with purified water.
6. pH of solution (3.0-3.5) was checked and stored at suitable conditions.

### Example—4

| Ingredient | mg/mL |
|---|---|
| Enalapril maleate | 1.00 |
| Citric acid anhydrous | 1.82 |
| Trisodium citrate | 0.15 |
| Sucralose | 0.70 |
| Potassium sorbate | 1.00 |
| Mixed berry flavor | 0.36 |
| Purified water | q.s |

### Brief Manufacturing Process:

1. Nitrogen gas was purged into a part of purified water for 30 minutes.
2. Potassium sorbate was added to step 1 and continuously stirred till clear solution was formed.
3. Citric acid followed by trisodium citrate was added to part of purified water and continuously stirred till clear solution was formed.
4. Step 2 and step 3 were continuously stirred till clear solution was formed.
5. Sucralose and flavor were added to step 4 and continuously stirred till clear solution was formed.
6. Enalapril maleate was added to step 5 and continuously stirred till clear solution was formed and final volume was made with purified water followed by nitrogen purging for 30 minutes.
7. pH of solution (3.0-3.5) was checked and stored at suitable conditions.

TABLE 2
Results of stability evaluation of Enalapril oral
liquid prepared from Example 4:

| | | | Example 4 | | |
|---|---|---|---|---|---|
| | | | (Enalapril Maleate | | |
| | Epaned ® | | oral solution 1 mg/mL) | | |
| | 25° C./ | | | 25° C./ | 2-8° C. |
| Related | 60% RH | 2-8° C. | | 60% RH | 3 |
| Substances | 3 months | 3 months | Initial | 3 months | months |
| Enalaprilat | 2.200% | 0.393% | 0.062% | 1.970% | 0.285% |
| (Impurity-C) | | | | | |
| Enalapril | 0.625% | 0.042% | 0.040% | 0.620% | 0.040% |
| Diketopiperazine | | | | | |
| (Impurity-D) | | | | | |
| Total impurities | 2.820% | 0.435% | 0.102% | 2.590% | 0.325% |

### Example—5

| Ingredient | mg/mL |
|---|---|
| Enalapril maleate | 1.00 |
| Citric acid anhydrous | 1.82 |
| Disodium hydrogen phosphate | 0.07 |
| Sucralose | 0.70 |
| Sodium benzoate | 1.00 |
| Strawberry flavour | 0.5 |
| Purified water | q.s |

### Brief Manufacturing Process:

1. Sodium benzoate to part of purified water was added and continuously stirred till clear solution was formed.
2. Citric acid followed by disodium hydrogen phosphate was added to part of purified water and continuously stirred till clear solution was formed.
3. Step 1 and step 2 were continuously stirred till clear solution was formed.
4. Sucralose and flavor were added to step 3 and continuously stirred till clear solution was formed.
5. Enalapril maleate was added to step 4 and continuously stirred till clear solution was formed and final volume was made with purified water.
6. pH of solution (3.0-3.5) was checked and stored at suitable conditions.

We claim:

1. A stable liquid composition for oral administration comprising 1 mg/mL of enalapril maleate and a buffer comprising citric acid in an amount of 1 mg/mL to 3 mg/mL and disodium hydrogen phosphate in an amount of 0.01 mg/mL to 0.3 mg/mL.

2. The composition of claim 1, is in the form of solution.

3. The composition of claim 1, further comprises a preservative selected from sodium benzoate, potassium sorbate, sorbic acid, methyl paraben and propyl paraben.

4. A stable liquid composition for oral administration comprising 1 mg/mL enalapril maleate and a buffer comprising citric acid in an amount of 1 mg/mL to 3 mg/mL and disodium hydrogen phosphate in an amount of 0.01 mg/mL to 0.3 mg/mL, wherein the composition has less than 1.5% of total impurities after storage for 15 months at 2-8° C.

5. The composition of claim 4, comprises 1 mg/mL enalapril maleate and a buffer comprising 1.82 mg/mL citric acid and upto 0.15 mg/mL disodium hydrogen phosphate.

6. The composition of claim 4, wherein total impurities include enalaprilat, enalapril diketopiperazine or both.

7. The composition of claim 4, comprises less than 1% of enalaprilat impurity and less than 0.2% of enalapril diketopiperazine impurity after storage for 15 months at 2-8° C.

8. A stable liquid composition for oral administration comprising 1 mg/mL enalapril maleate and a buffer comprising citric acid in an amount of 1 mg/mL to 3 mg/mL and disodium hydrogen phosphate in an amount of 0.01 mg/mL to 0.3 mg/mL; wherein the composition has less than 5% of total impurities after storage for 6 months at 25° C.

9. The composition of claim 7, wherein total impurities include enalaprilat, enalapril diketopiperazine or both.

10. The method of treating hypertension, heart failure and asymptomatic left ventricular dysfunction comprising administering to the patient the composition of claim 1.
